(19) United States
(12) Patent Application Publication
(10) Pub. No.: US 2022/0139548 A1
(43) Pub. Date: May 5, 2022

(54) NURSING ASSISTANCE DEVICE, NURSING ASSISTANCE METHOD, AND RECORDING MEDIUM

(71) Applicant: NEC Corporation, Minato-ku, Tokyo (JP)

(72) Inventors: Yuki KOSAKA, Tokyo (JP); Masahiro KUBO, Tokyo (JP); Yuan LUO, Tokyo (JP); Shigemi KITAHARA, Tokyo (JP)

(73) Assignee: NEC Corporation, Minato-ku, Tokyo (JP)

(21) Appl. No.: 17/285,224

(22) PCT Filed: Oct. 15, 2019
(86) PCT No.: PCT/JP2019/040475

§ 371 Date: Jan. 7, 2022

(30) Foreign Application Priority Data
Oct. 23, 2018 (JP) 2018-199528

(51) Int. Cl. G16H 40/67; G06N 5/02; G06F 16/338; G16H 40/20; G16H 10/60

(52) U.S. Cl. CPC G16H 40/67; G06N 5/025; G16H 10/60; G16H 40/20; G06F 16/338

(57) ABSTRACT

A nursing assistance device includes: a handling alert generation unit that generates a handling alert related to nursing based on nursing information and a nursing handling rule of a patient; and a handling alert output unit that outputs the handling alert to an output destination device related to a nursing attendant of the patient.

### TECHNICAL FIELD

The present invention relates to a nursing assistance device, a nursing assistance method, and a recording medium.

### BACKGROUND ART

Work of recording nursing information is very important in nursing work for a patient. All important matters related to nursing need to be recorded without omission. Here, nursing information refers to general information that should be understood by a nurse in nursing a patient such as a condition of the patient, nursing content for the patient, and a change in the condition of the patient in the nursing work for the patient. A plurality of nurses involved in caring for a patient can understand the condition of the patient and can perform appropriate care for the patient based on the understanding by confirming the nursing information.

Here, technologies related to nursing information are disclosed in Patent Documents 1 to 4.
Patent Document 1 discloses a technology capable of easily grasping a condition of an observation target person from observation results accumulated for the observation target person.
Patent Document 2 discloses a technology that enables decision of what a nurse needs to do for a group of patients in a specific time or a specific time range in addition to decision of what a nurse needs to do for a specific patient.
Patent Document 3 discloses a technology for displaying information requesting a behavior by health care personnel of a patient.
Patent Document 4 discloses a technology for presenting useful knowledge in a case where a nurse performs treatment for a patient.

### PRIOR ART DOCUMENTS

### Patent Documents

[Patent Document 1] PCT International Publication No. WO2015/125810
[Patent Document 2] Published Japanese Translation No. 2017-513125 of the PCT International Publication
[Patent Document 3] Japanese Unexamined Patent Application, First Publication No. 2016-167307
[Patent Document 4] Japanese Unexamined Patent Application, First Publication No. 2006-285565

### SUMMARY OF THE INVENTION

### Problem to be Solved by the Invention

Incidentally, the above-mentioned nursing information is often not sufficiently utilized, and a technology for efficiently notifying a user such as a nurse of information for handling a patient by a nursing attendant by utilizing the nursing information is required.

An example object of the present invention is to provide a nursing assistance device, a nursing assistance method, and a recording medium that solve the above-mentioned problems.

### Means for Solving the Problem

According to a first example aspect of the present invention, a nursing assistance device includes: a handling alert generation unit that generates a handling alert related to nursing based on nursing information and a nursing handling rule of a patient; and a handling alert output unit that outputs the handling alert to an output destination device related to a nursing attendant of the patient.

According to a second example aspect of the present invention, a nursing assistance method includes: generating a handling alert related to nursing based on nursing information and a nursing handling rule of a patient; and outputting the handling alert to an output destination device related to a nursing attendant of the patient.

According to a third example aspect of the present invention, a recording medium stores a program for causing a computer to execute: generating a handling alert related to nursing based on nursing information and a nursing handling rule of a patient; and outputting the handling alert to an output destination device related to a nursing attendant of the patient.

### Effect of the Invention

According to an example embodiment of the present invention, it is possible to provide a nursing assistance device that efficiently notifies a user of information for handling a patient of a nursing attendant by utilizing nursing information.

### BRIEF DESCRIPTION OF THE DRAWINGS

FIG. 1 is a diagram showing an outline of a nursing assistance system according to an example embodiment of the present invention.

FIG. 2 is a diagram showing a hardware configuration of a nursing assistance device according to the example embodiment of the present invention.

FIG. 3 is a functional block diagram of the nursing assistance device according to the example embodiment of the present invention.

FIG. 4 is a first diagram showing a data example stored in the nursing assistance device according to the example embodiment of the present invention.

FIG. 5 is a second diagram showing the data example stored in the nursing assistance device according to the example embodiment of the present invention.

FIG. 6 is a third diagram showing the data example stored in the nursing assistance device according to the example embodiment of the present invention.

FIG. 7 is a first diagram showing a processing flow of the nursing assistance device according to the example embodiment of the present invention.

FIG. 8 is a second diagram showing the processing flow of the nursing assistance device according to the example embodiment of the present invention.

FIG. 9 is a third diagram showing the processing flow of the nursing assistance device according to the example embodiment of the present invention.

FIG. 10 is a diagram showing a first example of a UI image according to the example embodiment of the present invention.

FIG. 11 is a diagram showing a second example of the UI image according to the example embodiment of the present invention.

FIG. 12 is a diagram showing a third example of the UI image according to the example embodiment of the present invention.

FIG. 13 is a diagram showing a fourth example of the UI image according to the example embodiment of the present invention.

FIG. 14 is a diagram showing a fifth example of the UI image according to the example embodiment of the present invention.

FIG. 15 is a diagram showing a sixth example of the UI image according to the example embodiment of the present invention.

FIG. 16 is a diagram showing a nursing assistance device according to another example embodiment of the present invention.

### EXAMPLE EMBODIMENT

Hereinafter, a nursing assistance device 1 according to an example embodiment of the present invention will be described with reference to the drawings.

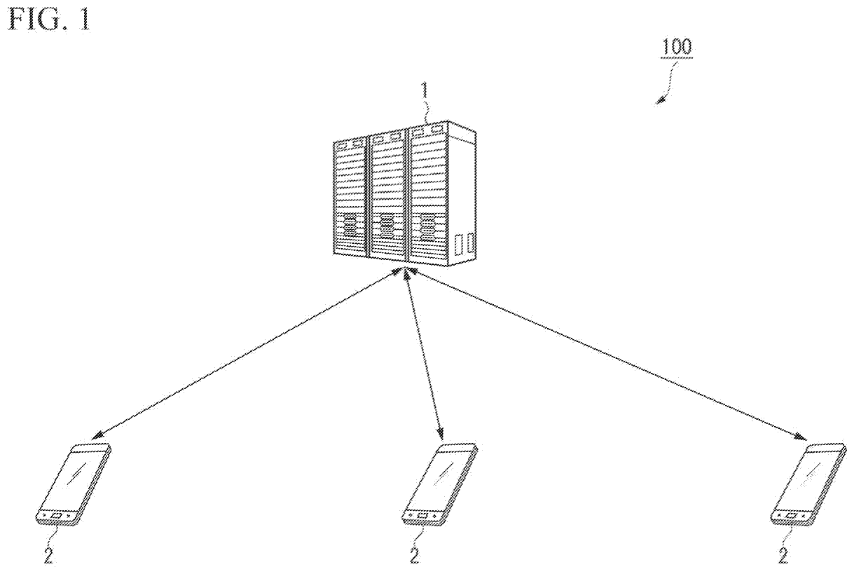

FIG. 1 shows an outline of a nursing assistance system 100 having the nursing assistance device 1 according to the present example embodiment.

The nursing assistance device 1 is installed in a hospital or the like. The nursing assistance device 1 may be installed outside the hospital. The nursing assistance device 1 is communicably connected with terminals 2 such as smartphones. The nursing assistance system 100 is constituted by the nursing assistance device 1 and the terminals 2. The terminal 2 is owned by, for example, a nurse or a doctor. The terminal 2 may be a PC or the like instead of a mobile terminal such as a smartphone.

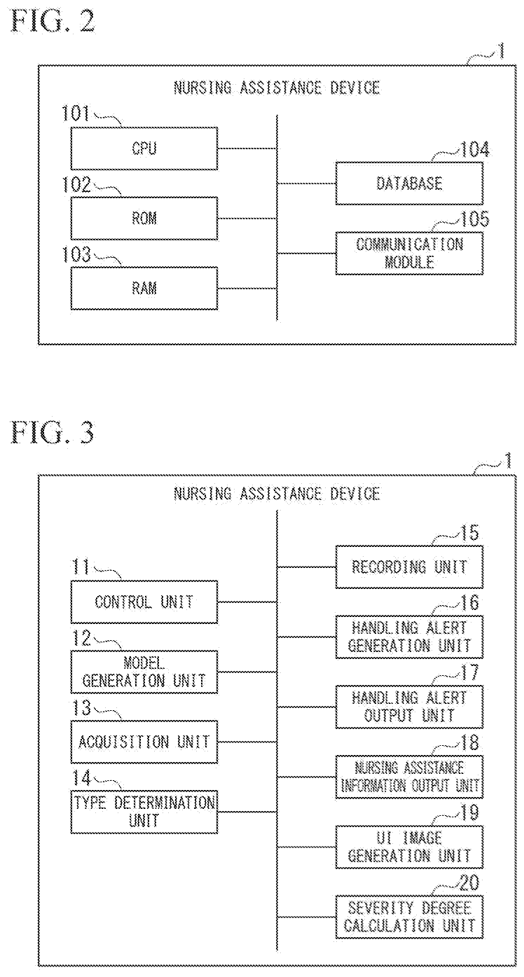

FIG. 2 is a diagram showing a hardware configuration of the nursing assistance device 1.

As shown in FIG. 2, the nursing assistance device 1 is a computer that includes hardware such as a central processing unit (CPU) 101, a read only memory (ROM) 102, a random access memory (RAM) 103, a database 104, and a communication module 105.

FIG. 3 is a functional block diagram of the nursing assistance device 1.

The nursing assistance device 1 executes a nursing assistance program. Accordingly, the nursing assistance device 1 has functions of a control unit 11, a model generation unit 12, an acquisition unit 13, a type determination unit 14, a recording unit 15, a handling alert generation unit 16, a handling alert output unit 17, a nursing assistance information output unit 18, a UI image generation unit 19, and a severity degree calculation unit 20.

The control unit 11 controls the other functional units.

The model generation unit 12 generates a record type determination model that specifies a record type for each nursing type based on past nursing information of a plurality of patients whose nursing types match each other. The record type determination model includes a content determination model and a SOAP determination model, which will be described in detail later.

The acquisition unit 13 acquires input information received from the terminal 2.

The type determination unit 14 determines at least a record type of the input information received from the terminal 2.

The recording unit 15 records, as nursing information, the input information associated with at least the record type.

The handling alert generation unit 16 generates a handling alert regarding nursing based on the nursing information and a nursing handling rule of a patient.

The handling alert output unit 17 outputs a handling alert to an output destination device associated with a nursing attendant of the patient. In the present example embodiment, the output destination device is the terminal 2.

The nursing assistance information output unit 18 outputs nursing assistance information related to the input information of the patient to the terminal 2 based on the acquisition of designation information for designating an identifier of the patient and past input information regarding the patient from the terminal 2.

The UI image generation unit 19 generates a UI image to be output to the terminal 2.

The severity degree calculation unit 20 calculates a severity degree of the patient.

The nursing assistance device 1 according to the present example embodiment generates the handling alert regarding the nursing based on the nursing information and the nursing handling rule of the patient, and outputs the handling alert to the terminal 2 associated with the nursing attendant of the patient.

By such processing, the nursing assistance device 1 transmits the handling alert to the terminal 2 with respect to a certain patient of the nursing information specified based on the nursing handling rule and thereby it is possible to efficiently notify a user such as a nurse or a doctor of awareness for handling the patient.

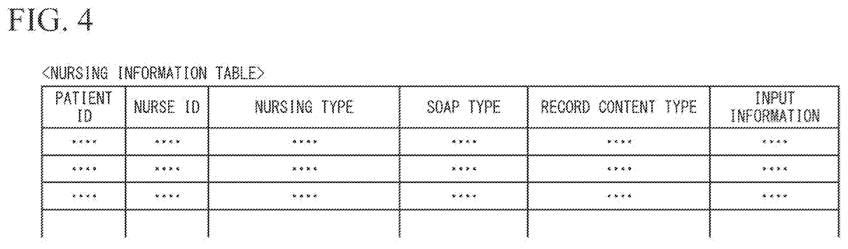

FIG. 4 is a first diagram showing a data example (nursing information table) stored in the nursing assistance device 1.

The nursing assistance device 1 records the nursing information associated with a patient ID, a nurse ID, a nursing type, a SOAP type, a record content type, input information, and the like in the nursing information table based on the acquisition of the input information received from the terminal 2. The nursing information table is recorded in the database 104. As an example, the nursing type indicates a type of nursing corresponding to a disease of the patient such as “fracture” or “stroke”. The nursing type may be information indicating a different type for each patient attribute such as a gender or an age group of the patient in addition to the type of the nursing corresponding to the disease of the patient. The SOAP type indicates information for specifying that the input information is any type of “Subjective (subjective information)”, “Objective (objective information)”, “Assessment (assessment information)”, and “Plan (plan, treatment information)”. The record content type is information for specifying that the input information is any state of the patient. The input information is main information that should be recorded as the nursing information input by the user such as the nurse or the doctor via the terminal 2. The nursing information may not include the nursing type.

The SOAP type and the record content type will be described below.

The input information received from the terminal 2 is information regarding the patient. This input information is recorded as the nursing information. The nursing assistance device 1 specifies the record type of the input information in a case where the input information is recorded as the nursing information. In the present example embodiment, the record type is roughly classified into a SOAP type and a content type. The record content type is information for identifying the content of the input information. SOAP is a word taken from the initials of “Subjective (subjective information)”, “Objective (objective information)”, “Assessment (assessment information)”, and “Plan (plan, treatment information)”, and indicates the types of these pieces of information. Hereinafter, the type of “Subjective” is written as S. The type of “Objective” is written as O. The type of “Assessment” is written as A. The type of “Plan” is written as P. “Subjective” indicates information obtained directly from the patient. “Objective” indicates information obtained from a physical examination and an inspection by the nurse. “Assessment” indicates information of assessment by the nurse or the doctor based on “Subjective” and “Objective”. “Plan” indicates a plan or a treatment performed as a result of “Assessment”.

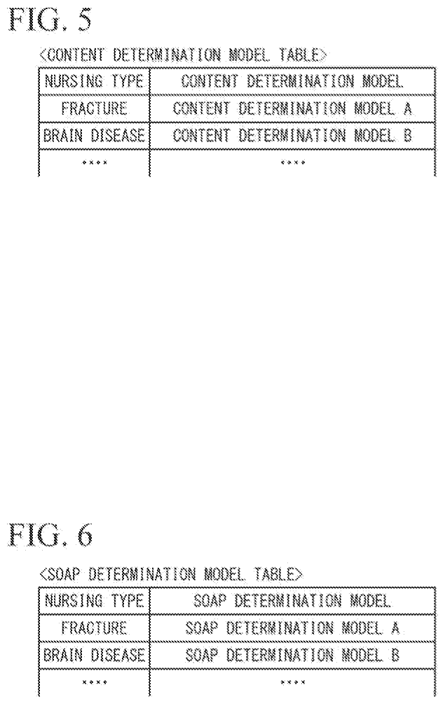

FIG. 5 is a second diagram showing a data example (content determination model table) stored in the nursing assistance device 1.

The content determination model is generated by the model generation unit 12 of the nursing assistance device 1 based on the pieces of past nursing information of the plurality of patients. The content determination model is provided for each nursing type. The content determination model is recorded in the database 104. The content determination model is a model for determining the record content type. The nursing assistance device 1 stores the content determination model table for specifying the content determination model corresponding to the nursing type in the database 104. The nursing assistance device 1 specifies the content determination model corresponding to the nursing type using this content determination model table, and determines the record content type of the input information by using the content determination model. In a case where the nursing type is not included in the nursing information, one content determination model may be recorded in the nursing assistance device 1, and the record content type of the input information may be determined by using this content determination model.

FIG. 6 is a third diagram showing a data example (SOAP determination model table) stored in the nursing assistance device 1.

The SOAP determination model is generated by the model generation unit 12 of the nursing assistance device 1 based on the pieces of past nursing information of the plurality of patients. The SOAP determination model is provided for each nursing type. The SOAP determination model is recorded in the database 104. The nursing assistance device 1 stores the SOAP determination model table for specifying the SOAP determination model corresponding to the nursing type. The nursing assistance device 1 specifies the SOAP determination model corresponding to the nursing type using this SOAP determination model table, and determines the SOAP type of the input information by using the SOAP determination model. In a case where the nursing type is not included in the nursing information, one SOAP determination model may be recorded in the nursing assistance device 1, and the record content type of the input information may be determined by using this SOAP determination model. For example, the nursing assistance device 1 determines which one of the record content types of “Subjective (subjective information)”, “Objective (objective information)”, “Assessment (assessment information)”, and “Plan (plan, treatment information)” the input information included in the nursing information is, by using the SOAP determination model.

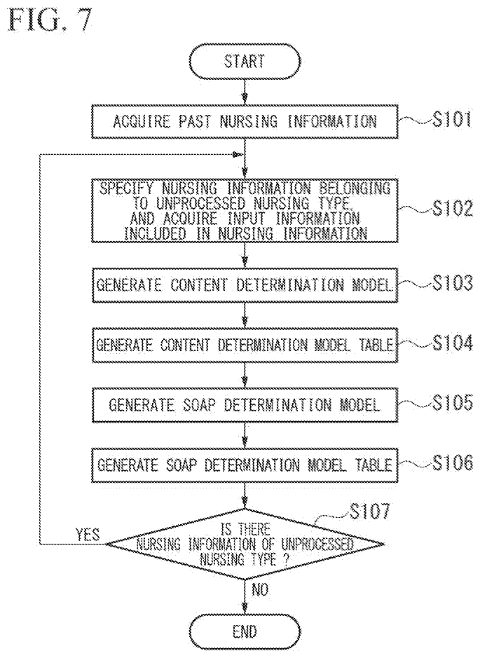

FIG. 7 is a first diagram showing a processing flow of the nursing assistance device 1.

The pre-processing of the nursing assistance device 1 will be described with reference to FIG. 7.

The nursing assistance device 1 acquires pieces of nursing information recorded in an electronic medical record database or the pieces of nursing information stored in the device itself in the past (step S101). The acquired nursing information includes the patient ID, the nurse ID, the nursing type, the SOAP type, the record content type, the input information, and the like. The input information is information input by the user with their voice by using the terminal 2 in the past. The voice from the voice input is converted into characters. The input information converted into the characters is recorded as the nursing information in the electronic medical record database, the database 104, or the like. The input information may be information input as characters on the terminal 2. The model generation unit 12 of the nursing assistance device 1 acquires the pieces of nursing information recorded in the past. For example, in the database 104, words each constituting the input information included in the nursing information in advance, a class of the record content type or the SOAP type indicated by the input information, and a character string representing the class are given for each nursing type, and are included in the nursing information.

The nursing assistance device 1 generates the record type determination model. The record type determination model includes the content determination model and the SOAP determination model.

The model generation unit 12 of the nursing assistance device 1 specifies the pieces of nursing information belonging to an unprocessed nursing type from among all the pieces of acquired nursing information, and acquires the pieces of input information included in the pieces of nursing information (step S102). The model generation unit 12 also acquires pieces of paired information of all the pieces of input information and the record content types corresponding to the pieces of input information from the pieces of nursing information acquired in step S102 belonging to the unprocessed nursing type. The model generation unit 12 generates, based on the paired information of the input information and the record content types corresponding to the pieces of input information, the content determination model that determines which class of a plurality of record content type classes input information included in newly acquired nursing information belongs to (step S103). More specifically, the model generation unit 12 converts the input information included in the newly acquired nursing information into a vector in which a plurality of words included in the input information is represented by directions and sizes in a plurality of dimensions. The model generation unit 12 generates the content determination model that uses, as an input, the vector obtained by converting the input information based on each word constituting the input information included in the newly acquired nursing information, and consequently outputs any type of the record content types. The content determination model is a model for determining the record content type class in which each word appears unevenly and frequently among the record content type classes based on each word obtained from an analysis result of a sentence based on the pieces of input information. As an example, the content determination model is a model for determining the record content type class in which, in a case where the word is expressed by any vector, the vector appears unevenly and frequently based on the vector indicating the word. The model generation unit 12 generates the content determination model table (FIG. 5) representing identification information of the content determination model corresponding to the nursing type, and records it in the database 104 (step S104).

The model generation unit 12 also acquires, from the pieces of nursing information acquired in step S102 belonging to the unprocessed nursing type, paired information of all the pieces of input information and any SOAP type of S/O/A/P corresponding to the input information. The model generation unit 12 generates the SOAP determination model that determines which type f a plurality of SOAP types of S/O/A/P of the SOAP type the input information included in the newly acquired nursing information belongs to, based on the paired information of the input information and the SOAP type corresponding to the input information (step S105). More specifically, the model generation unit 12 converts the input information included in the newly acquired nursing information into a vector in which a plurality of words included in the input information is represented by directions and sizes in a plurality of dimensions. The model generation unit 12 generates the SOAP determination model that uses, as the input, the vector obtained by converting the input information based on the word constituting the input information included in the newly acquired nursing information and consequently outputs any type among the SOAP types. The model generation unit 12 generates the SOAP determination model table (FIG. 6) representing identification information of the SOAP determination model corresponding to the nursing type, and records it in the database 104 (step S106). The model generation unit 12 determines whether or not there are the pieces of nursing information of the unprocessed nursing type (step S107). The model generation unit 12 repeats the processing from step S102 in a case where there are the pieces of nursing information of the unprocessed nursing type.

The pre-processing of the nursing assistance device 1 is ended by the above processing. The content determination model and the SOAP determination model may be retained by the nursing assistance device 1 in advance. The content determination model and the SOAP determination model may be generated by using an algorithm of a general discriminator such as a Support Vector Machine.

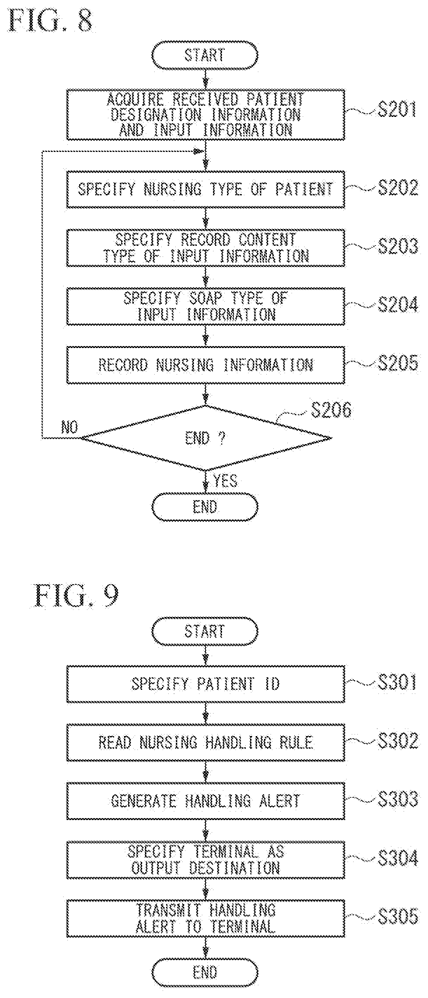

FIG. 8 is a second diagram showing a processing flow of the nursing assistance device 1.

The nursing assistance device 1 receives the input of the input information in a state where the content determination model table and the SOAP determination model table are generated as described above. The nursing assistance device 1 performs the generation and output processing of the nursing handling alert based on the nursing information and the nursing handling rule of the patient.

First, the user such as the nurse or the doctor activates a nursing assistance application program recorded in the terminal 2. Accordingly, the terminal 2 has functions of outputting a user interface (UI) screen of nursing assistance business to a display of the terminal 2, inputting the character information by using the UI screen, acquiring the voice input based on an operation on the UI screen, transmitting the input information to the nursing assistance device 1, and outputting various kinds of information received from the nursing assistance device 1 to the UI screen.

The user inputs any one or a plurality of pieces of information of the information (S) directly obtained from the patient, the information (O) obtained from the physical examination and the inspection by the nurse, the information (A) of the assessment of the patient, and the information (P) indicating the performed plan or treatment by using the terminal 2 at an appropriate timing. The input may be performed by voice, or may be input by character input using the input function provided in the terminal 2. At this time, the user designates necessary patient designation information such as the patient ID and the nurse ID by using the UI screen. Alternatively, the necessary patient designation information such as the patient ID and the nurse ID may be input by voice. The terminal 2 transmits the patient designation information and the input information to the nursing assistance device 1.

The terminal 2 may have a function of automatically starting an operation using the nursing assistance application program based on communication with a predetermined communication device. For example, the terminal 2 receives a signal transmitted from a signal transmission device which is one aspect of the communication device located beside a bed of the patient. In a case where the received signal is a predetermined signal, the terminal 2 may have each function of the terminal 2 by automatically activating the nursing assistance application program. In this case, as an example, the terminal 2 acquires the voice of the user such as the nurse or the doctor and the voice of the patient from a microphone based on an operation of starting voice acquisition and ending voice acquisition of the user or the like. The terminal 2 may use the voice information as the input information and transmit the input information and the patient designation information to the nursing assistance device 1. Accordingly, since the terminal 2 omits the activating operation of the user, acquires the voice based on the start of the voice acquisition, and transmits the voice to the nursing assistance device 1, the user can input the input information quickly and without effort from the user during the nursing work. The terminal 2 may automatically acquire the voice of the user or the patient without the operation input for starting the voice acquisition. The above-mentioned signal transmission device may add the patient ID in the patient designation information to the signal and may transmit the signal, and the terminal 2 that receives the signal may detect the patient ID included in the signal.

The acquisition unit 13 acquires the input information and the patient designation information received by the nursing assistance device 1 (step S201). The acquisition unit 13 outputs the input information to the type determination unit 14. The type determination unit 14 reads and specifies the nursing type of the patient recorded in the database 104 in advance in association with the patient ID included in the patient designation information (step S202). This nursing type is determined according to the patient attributes such as a gender, an age, and a disease of the patient. The type determination unit 14 specifies the content determination model based on the information recorded in the content determination model table (FIG. 5) and the nursing type of the patient. The type determination unit 14 acquires a model formula of the content determination model from a storage unit or the like. The type determination unit 14 performs morphological analysis on the input information, and inputs the input information vector generated by the obtained words and the frequencies thereof into the content determination model. As a result of the input, the type determination unit 14 specifies the record content type of the input information by the determination processing using the content determination model (step S203).

The type determination unit 14 also specifies the SOAP determination model based on the information recorded in the SOAP determination model table and the nursing type of the patient. The type determination unit 14 acquires a model formula of the SOAP determination model from the storage unit or the like. The type determination unit 14 performs morphological analysis on the input information, and inputs the input information vector generated by the obtained words and the frequencies thereof into the SOAP determination model. As a result of the input, the type determination unit 14 specifies the SOAP type of the input information by the determination processing using the SOAP determination model (step S204). The type determination unit 14 outputs the record content type and the SOAP type of the input information acquired in step S201 to the recording unit 15.

The acquisition unit 13 outputs the patient designation information, the input information, the nursing type, and the record content type to the recording unit 15. The recording unit 15 records the nursing information associated with the patient ID, the nurse ID, the nursing type, the SOAP type, the record content type, and the input information included in a set type in the nursing information table (step S205).

The acquisition unit 13 and the type determination unit 14 repeat the above processing whenever the nursing assistance device 1 receives the input information received from the terminal 2. The control unit 11 determines whether to end the processing of recording the nursing information (step S206), and repeats the processing of recording the nursing information until it is determined to end. Accordingly, the input information of a hospital patient is accumulated as the nursing information in the nursing information table 4.

In a case where certain specific nursing information is recorded, the nursing assistance device 1 starts processing of generating the handling alert regarding the nursing based on the nursing information and the nursing handling rule and outputting the handling alert to a predetermined output destination device. The handling alert is an alert issued to the user such as the nurse or the doctor, and is an alert prompting some treatment (handling) for the patient. The handling alert may be, for example, an alert for the nursing attendant such as the nurse forgetting to prescribe medicine for the hospital patient, or an alert prompting to stop handling in a case where an interval between a previous administration point in time of the medicine and a next administration point in time of the medicine is less than a predetermined time. This processing is an example of an aspect in which the handling alert generation unit 16 generates the handling alert when, in a case where specific information is included in the nursing information and the nursing handling rule indicates medicine information, a registration point in time at which the nursing information is recorded and a next registration point in time of the nursing information of this medicine information are within a predetermined time. The nursing assistance device 1 performs the following processing in parallel with the processing of recording the nursing information described above.

FIG. 9 is a third diagram showing a processing flow of the nursing assistance device 1.

The acquisition unit of the nursing assistance device 1 outputs the patient designation information and the input information acquired in step S201 to the handling alert generation unit 16. The handling alert generation unit 16 specifies the patient ID from the patient designation information (step S301). The handling alert generation unit 16 acquires the nursing type stored in the database 104 in advance in association with the patient ID. The nursing type is indicated by a number or an ID for specifying a disease of the patient, a type of treatment or care required for the patient based on a condition of the patient including the disease or the like, a hospital stay period, and the like. The handling alert generation unit 16 reads the nursing handling rule stored in the database 104 in advance in association with the nursing type based on the nursing type (step S302). The handling alert generation unit 16 may read the nursing handling rule stored in advance in the database 104 in association with any one of the record content type and the SOAP type or a combination of both types in advance based on the record content type specified in step S202 based on the input information and the SOAP type specified in step S203. The nursing handling rule may be information indicating a handling rule of the nursing corresponding to one or a plurality of the nursing type or the record content type or the SOAP type.

In a case where the acquired input information is the input information specified by the nursing handling rule, the handling alert generation unit 16 generates a predetermined handling alert specified by the nursing handling rule (step S303). The handling alert generation unit 16 outputs the generated handling alert to the handling alert output unit. The handling alert output unit 17 specifies the terminal 2 serving as the output destination based on the nurse ID included in the patient designation information acquired in step S201 (step S304). The handling alert output unit 17 transmits the handling alert to the specified terminal 2 (step S305).

The terminal 2 is communicably connected with the nursing assistance device 1 by the operation using the nursing assistance application program, and outputs the UI image generated by the UI image generation unit 19 of the nursing assistance device 1 to the display. In a case where the handling alert is received, the terminal 2 updates the UI image based on each piece of information included in the handling alert. Accordingly, the user such as the nurse or the doctor is notified of the information of the handling alert.

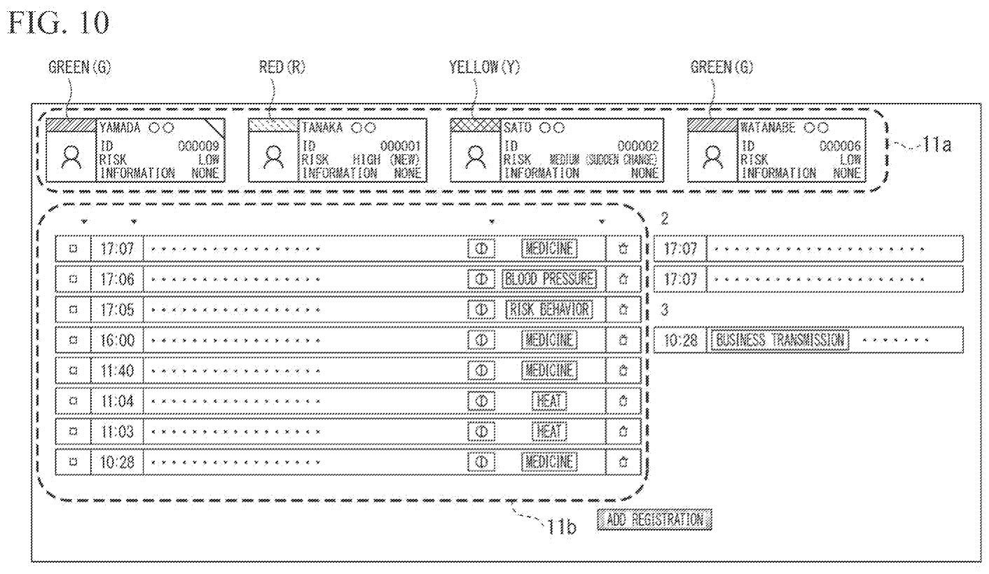

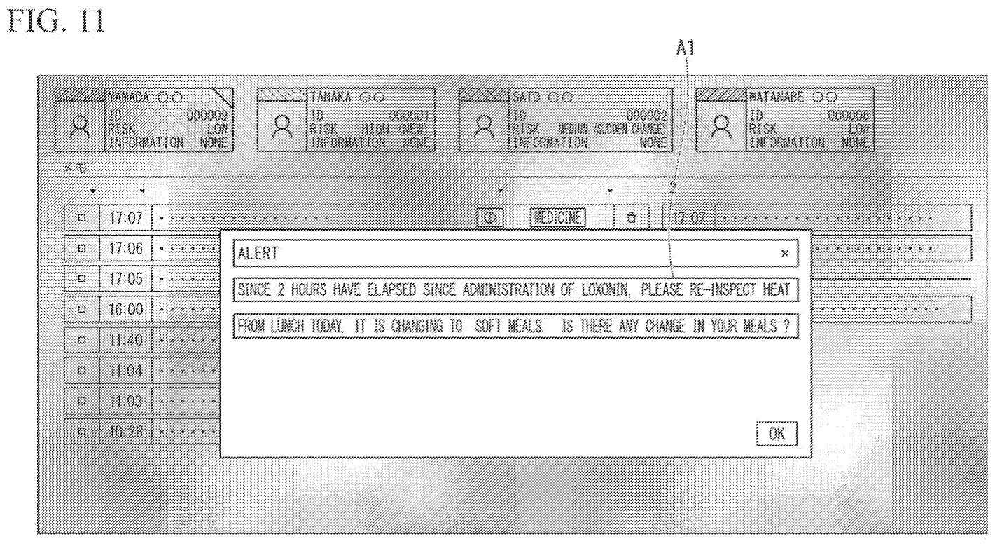

FIG. 10 is a diagram showing a first example of the UI image.

The UI image generation unit 19 of the nursing assistance device 1 generates the UI image as shown in FIG. 10 as an example, and outputs it to the terminal 2. The UI image shown in FIG. 10 is an image to be transmitted by the nursing assistance device 1 to the terminal 2, such as a PC, which is capable of being disposed on a desktop and has a relatively large display size. The UI image may include at least a list display region 11a of patients cared for by the user using the terminal 2 and a nursing information display region 11b for displaying the nursing information of a designated patient among these patients.

FIG. 11 is a diagram showing a second example of the UI image.

The handling alert generation unit 16 uses the nursing handling rule defined to output the alert in a case where the input information indicates a predetermined record content type and a predetermined SOAP type when generating the handling alert in step S303. In this case, the handling alert generation unit 16 acquires the determination result of the record content type of the input information and the determination result of the SOAP type from the type determination unit 14. In a case where both the acquired record content type and SOAP type match each type included in the nursing handling rule, the handling alert generation unit 16 generates a predetermined handling alert described in the nursing handling rule.

As a specific example, a case will be described in which, as a nursing handling rule, it is defined that, in a case where the character string representing the class of the record content type is “medicine”, the specific medicine name is included in the input information, and the SOAP type is “P (treatment information)”, the character string prompting to perform a body temperature measurement will be transmitted after two hours has passed from a prescription point in time of the medicine indicated by the medicine. In this case, the handling alert generation unit 16 determines, based on the input information, whether the record content type is “medicine”, the specific medicine name is included in the input information, and the SOAP type is “P (treatment information)”. In a case where it is determined, based on the input information, that the record content type is “medicine”, the specific medicine name (“Loxonin” in the example of FIG. 11) is included in the input information, and the SOAP type is “P (treatment information)”, the handling alert generation unit 16 detects the prescription point in time included in the input information. The handling alert generation unit 16 generates, 2 hours (a predetermined time) after the prescription point in time, the handling alert including the character string prompting to perform the body temperature measurement (the measurement of the patient) to be performed, based on the detected prescription point in time. The handling alert output unit 17 specifies the terminal 2 based on the nursing ID included in the patient designation information received from the terminal 2 together with the input information, and transmits, 2 hours after a point in time registered as the nursing information of the input information, the handling alert including the character string prompting to perform the body temperature measurement on the terminal 2. In a case where this handling alert is received, the terminal 2 generates and outputs a character string (A1) prompting to perform the body temperature measurement included in the handling alert to the UI image (FIG. 11) of the display of the terminal 2.

As another specific example, a case will be described in which, as a nursing handling rule, it is defined that, in a case where the record content type is “medicine”, the specific medicine name is included in the input information, and the SOAP type is “P (treatment information)”, the character string prompting to perform the body temperature measurement will be transmitted after 3 hours has passed since the treatment (medication) is performed by the administration of the specific medicine. In this case, in a case where it is determined, based on the input information, that the record content type is “medicine”, the specific medicine name is included in the input information, and the SOAP type is “P (treatment information)”, the handling alert generation unit 16 generates the handling alert including the character string prompting to perform the body temperature measurement after 3 hours has passed. Even though the input information included in the nursing information indicates that the record content type is “medicine”, the specific medicine name is included in the input information, and the SOAP type is “P (treatment information)”, in a case where the input information indicating that the body temperature measurement is performed before 3 hours elapse from the recording point in time of the nursing information is registered, the handling alert generation unit 16 may not generate the handling alert. The handling alert output unit 17 specifies the terminal 2 based on the nursing ID included in the patient designation information received from the terminal 2 together with the input information, and transmits the handling alert including the character string prompting to perform the body temperature measurement 3 hours after the point in time registered as the nursing information of the input information in the terminal 2. In a case where this handling alert is received, the terminal 2 generates and outputs a character string (A1) prompting to perform the body temperature measurement included in the handling alert to the UI image (FIG. 11) of the display of the terminal 2. Accordingly, the user such as the nurse can perform the body temperature measurement after a predetermined time elapses from the administration of the medication without forgetting about the measurement. The processing of the handling alert generation unit 16 is an example of an aspect in which information regarding a treatment included in the nursing information is specified and a handling alert prompting to perform a predetermined treatment is generated by using a nursing handling rule indicating the predetermined treatment corresponding to the treatment. The predetermined treatment may be a future treatment.

FIGS. 10 and 11 show, as an example, the UI image to be transmitted to the terminal 2, such as a PC, which is capable of being disposed on a desktop and has a relatively large display size. However, the nursing assistance device 1 may generate a UI image to be transmitted to the portable terminal 2 such as a smartphone by changing a size thereof and arrangement of display information, and may transmit the UI image to the terminal 2.

The handling alert generation unit 16 may constantly monitor the nursing information and may generate the handling alert at a timing at which it is determined that the handling alert is generated based on the nursing information in addition to generation of the handling alert at a timing immediately after the input information is acquired. For example, the handling alert generation unit 16 reads the nursing information as the nursing information is accumulated, and specifies the patient ID of the patient included in the nursing information. The handling alert generation unit 16 selects the patient ID of the first patient and reads medical record information recorded in the database 104 in association with the patient ID.

The handling alert generation unit 16 outputs the medical record information to the severity degree calculation unit 20 and instructs it to calculate a severity degree of the patient. The severity degree calculation unit 20 calculates the severity degree of the patient based on the medical record information of the patient. The medical record information includes a treatment and care history for the patient. The treatment and care history includes a disease type for identifying the disease of the patient, a nursing type for identifying types of the treatment and care required for the patient based on the condition of the patient including the disease and the like for each day of the hospital stay period, a surgery mode of the doctor based on the disease, a disease type occurring during the hospital stay period and the date on which such a disease occurs, and condition information such as a body temperature of each day during the hospital stay period of the patient and the number of times of urination and defecation. The treatment and care history may include other information. The severity degree calculation unit 20 may calculate the severity degree of the patient by inputting, for example, numerical values related to the treatment and care history included in the medical record information into a severity degree calculation formula. The severity degree calculation unit 20 determines whether a level of the severity degree of the patient is any one of three levels, that is, “high”, “medium”, and “low” by comparing the severity degree with threshold values. The severity degree calculation unit 20 outputs the severity degree of the patient to the handling alert generation unit 16.

The handling alert generation unit 16 generates a handling alert corresponding to the level of the severity degree of the patient. The level of the severity degree of the patient is set, for example, such that a level of a severity degree of a patient whose body temperature is higher than a control value instructed by the doctor or a level of a severity degree of a patient within 24 hours from a surgery end point in time is “high”. For example, in the case of a patient who also has a disease that occurred during the hospital stay period, the level of the severity degree of the patient is set to “medium”. The levels of the severity degrees of other patients are set to “low”. The handling alert is information for designating a color for each patient in the list display region 11a of the patients on the UI image, for example. The handling alert also includes the patient ID. The handling alert output unit 17 transmits this handling alert to the terminal 2. Then, the terminal 2 specifies the patient corresponding to the patient ID included in the handling alert among the patients displayed in the list display region 11a of the patients on the UI image. The terminal 2 decides the color to be displayed for the patient based on the information for designating the color included in the handling alert, and displays the attributes (name, patient ID, and the like) of the patient with the color. As an example, in a case where the level of the severity degree of the patient is “high”, red (R) is set, in a case where the level of the severity degree of the patient is “medium”, yellow (Y) is set, and in a case where the level of the severity degree of the patient is “low”, green (G) is set. The terminal 2 may display the level of the severity degree of the patient indicated by the handling alert with a display aspect other than color.

The severity degree calculation unit 20 may acquire the medical record information of each patient from the database 104 at a predetermined time interval and may calculate the severity degree of each patient at each time interval. In a case where the severity degree of each patient calculated by the severity degree calculation unit 20 is acquired and the UI image for displaying a list of patients as shown in FIGS. 10 and 11 is generated, the UI image generation unit 19 may set a color corresponding to the severity degree of each patient, and may generate the UI image. Accordingly, the nurse who confirms the UI image transmitted to the terminal 2 can understand the severity degree of the patient at a glance.

The handling alert generation unit 16 may generate the handling alert prompting to perform a predetermined treatment by using the nursing handling rule indicating the predetermined treatment corresponding to biological information of the patient included in the nursing information.

For example, the handling alert generation unit 16 reads the nursing information as the nursing information is accumulated, and specifies the patient ID of the patient included in the nursing information. The handling alert generation unit 16 selects the patient ID of the first patient and reads medical record information recorded in the database 104 in association with the patient ID. The handling alert generation unit 16 acquires the nursing type recorded in the database 104 in association with the patient ID, and acquires the nursing handling rule associated with the nursing type from the database 104. It is assumed that the nursing handling rule is defined, for example, such that in a case where the body temperature is equal to or greater than a predetermined threshold value, a character string prompting the user such as the nurse to perform cooling for lowering the body temperature of the patient is transmitted. More specifically, a case will be described in which, as a specific example of this nursing handling rule, it is defined that, when an instruction of the doctor in a case where the body temperature is equal to or greater than a threshold value is described in the medical record information and the body temperature of the patient is equal to or greater than the threshold value, a character string notifying the user such as the nurse of information indicating an instruction of cooling by the doctor will be transmitted. In this case, the handling alert generation unit 16 acquires the biological information of the patient based on the medical record information of the patient. More specifically, the handling alert generation unit 16 acquires the body temperature of the patient measured during a predetermined period included in the medical record information, for example. In a case where the body temperature is equal to or greater than a predetermined threshold value during the predetermined period based on a change in the body temperature, the handling alert generation unit 16 generates a handling alert including a character string prompting to perform cooling. The handling alert output unit 17 transmits the handling alert to the terminal 2. Upon receiving this handling alert, the terminal 2 outputs a character string prompting to perform cooling included in the handling alert to the UI image (FIG. 11) of the display of the terminal 2. Accordingly, the user such as the nurse performs a cooling treatment as instructed based on the instruction received in advance. Accordingly, the user such as the nurse can quickly notice and execute the cooling treatment in a case where the body temperature of the patient rises.

In a case where the input information including the biological information of the patient is recorded as the nursing information, when the input information matches the nursing handling rule, the handling alert generation unit 16 may generate a handling alert prompting to perform a predetermined treatment.

A case will be described in which, as a specific example of this nursing handling rule, it is defined that, when an instruction of the doctor in a case where the body temperature is equal to or greater than a threshold value is described in the medical record information and the body temperature of the patient is equal to or greater than the threshold value, a character string notifying the user such as the nurse of information indicating an instruction of the doctor in the medical record information will be transmitted. In this case, the handling alert generation unit 16 acquires the threshold value of the increase in the body temperature included in the medical record information and the instruction information of the doctor. The handling alert generation unit 16 generates the handling alert including the character string of the instruction information of the doctor in a case where the body temperature is equal to or greater than the threshold value in a predetermined period based on the change in the body temperature of the patient based on the input information included in the nursing information. Moreover, the handling alert output unit 17 transmits the handling alert to the terminal 2. Upon receiving the handling alert, the terminal 2 outputs the character string indicating the instruction information of the doctor to the UI image (FIG. 11) of the display of the terminal 2. Accordingly, the user such as the nurse can perform a treatment corresponding to the instruction of the doctor at an early stage in a case where the body temperature of the patient has risen to be equal to or greater than the threshold value.

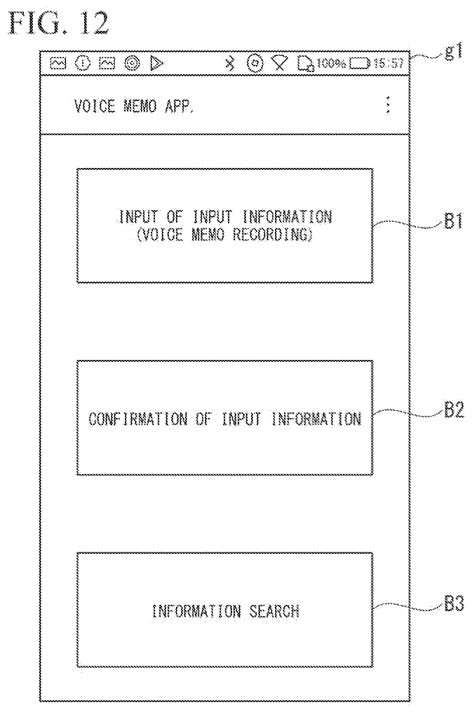

FIG. 12 is a diagram showing a third example of the UI image.

The UI image generation unit 19 of the nursing assistance device 1 generates, as another example, the UI image g1 as shown in FIG. 12 and outputs the UI image to the terminal 2. The UI image g1 shown in FIG. 12 is an image to be transmitted by the nursing assistance device 1 to the terminal 2, such as a smartphone, which is potable and has a relatively small display size. The UI image g1 shown in FIG. 12 shows an example of a case where an operation button B1 for the user of the terminal 2 to input the input information (voice memo recording), an operation button B2 for confirming the input information, and an operation button B3 for searching for information are displayed.

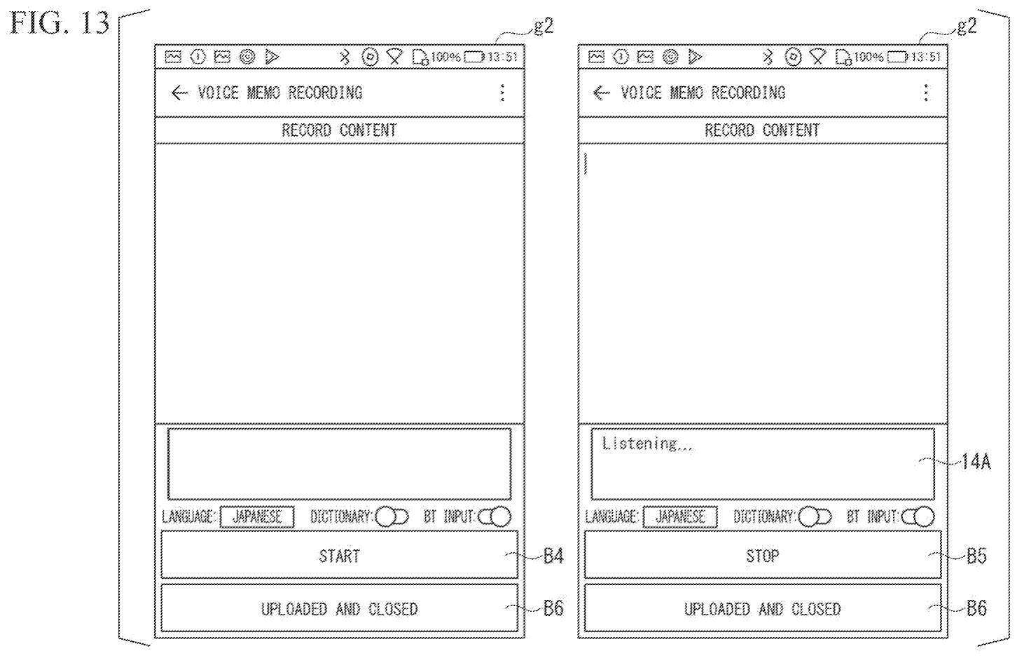

FIG. 13 is a diagram showing a fourth example of the UI image.

In the UI image g1 shown in FIG. 12, when the user of the terminal 2 presses the input button B1 of the input information, the terminal 2 outputs a UI image g2 shown in FIG. 13. Upon outputting the UI image g2, the terminal 2 waits for voice recording. A start (START) button B4 is displayed on the UI image g2 as shown in FIG. 13. In a case where the user performs a pressing operation on the start button B4, a recording function of the terminal 2 acquires voice which is one aspect of the input information via a microphone. Upon detecting a pressing operation of the start button B4 by the user, the terminal 2 displays a stop (STOP) button B5 on the UI image g2 instead of the start button B4. In a case where the user ends inputting the input information by the voice, the user performs a pressing operation on the stop button B5. Then, the terminal 2 stops the recording function. The terminal 2 may convert the acquired voice into characters and output the characters to an input information display region 14A of the UI image g2. An input information upload button B6 is displayed on the UI image g2. The user presses the upload button B6. The terminal 2 transmits, as input information, the character information converted from the input voice to the nursing assistance device 1.

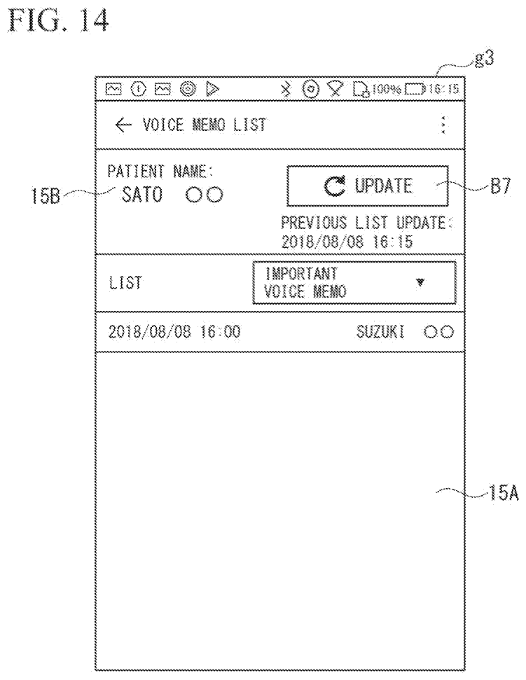

FIG. 14 is a diagram showing a fifth example of the UI image.

In the UI image g1 shown in FIG. 12, when the user of the terminal 2 presses the confirmation button B2 of the input information, the terminal 2 displays the patient selection list. The user selects a desired patient name in the patient selection list. The terminal 2 outputs a UI image g3 shown in FIG. 14 according to the selection. The UI image g3 displays a list display region 15A of the input information, a patient information display region 15B, an update button B7, and the like. The terminal 2 displays the name of the patient designated by the user in the patient information display region 15B. Upon detecting the selection of the name of the patient by the user, the terminal 2 transmits a nursing information list request including the patient ID of the patient to the nursing assistance device 1.

The nursing assistance device 1 receives the nursing information list request. The UI image generation unit 19 acquires the nursing information list request received by the nursing assistance device 1. The UI image generation unit 19 acquires the patient ID from the nursing information list request. The UI image generation unit 19 acquires at least the input information of the nursing information recorded in the database 104 in association with the patient ID. The UI image generation unit 19 generates a nursing information list including the acquired input information and transmits the nursing information list to the terminal 2. The UI image generation unit 19 may generate a nursing information list including only predetermined input information among the input information indicated by the nursing information acquired based on the patient ID. For example, the UI image generation unit 19 may generate the nursing information list including only the input information recorded in a time range from a current point in time to a point in time before a predetermined time. Alternatively, an importance flag may be attached to each piece of input information of the nursing information, they may be recorded in the database 104, and a nursing information list including only the input information to which the importance flag is attached may be generated. The terminal 2 displays one or a plurality of pieces of past input information included in the nursing information list in the list display region 15A. Accordingly, the user can browse the input information input in the past for the predetermined patient, and can grasp a past nursing status for the patient. Even if the patient who is a nursing information target displayed in the list display region 15A is not the patient in the charge of the user such as the nurse and the patient whose detailed information is unknown, a nursing status can be instantly grasped. Even if the patient is not the patient in charge, the user can provide quick and appropriate nursing based on the grasped information. The terminal 2 may perform voice output processing for reading the characters of the input information displayed in the list display region 15A of the input information. The user can instruct the terminal 2 to retransmit the nursing information list request by performing a pressing operation on the update button B7. That is, the terminal 2 that detects the pressing of the update button B7 transmits the nursing information list request to the nursing assistance device 1. Subsequent processing is the same as the above processing.

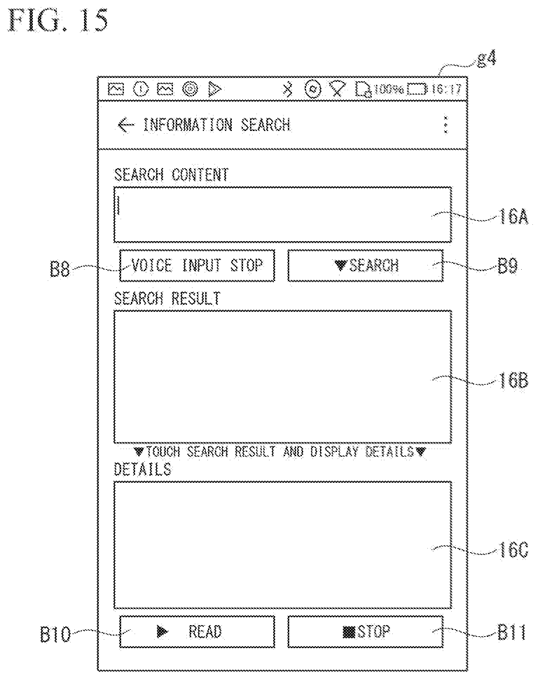

FIG. 15 is a diagram showing a sixth example of the UI image.

In the UI image g1 shown in FIG. 12, when the user of the terminal 2 presses the information search button B3 the terminal 2 displays the displayed patient selection list. The user selects the name of the desired patient in the patient selection list. Then, the terminal 2 outputs a UI image g4 shown in FIG. 15. The UI image g4 includes a search content input field 16A, a search result display field 16B, a detailed display field 16C, a voice input stop button B8, a search button B9, a reading button B10, a stop button B11, and the like.

The user inputs the search content. As an example, immediately after the UI image g4 is displayed, the terminal 2 turns on the recording function and automatically acquires the voice which is one aspect of the input information via the microphone. The terminal 2 analyzes the voice, converts the voice into character information, and outputs the character information to the search content input field 16A. Alternatively, the terminal 2 may directly acquire the character information with an operation of input means by a user and output the character information to the search content input field 16A. In a case where the user presses the search button B9, the terminal 2 transmits a search request including the character information included in the search content and the patient ID of the selected patient to the nursing assistance device 1.

The nursing assistance device 1 receives the search request. The nursing assistance information output unit 18 acquires the search request. The nursing assistance information output unit 18 acquires the patient ID and the character information of the search content that are included in the search request. The search content may include the record content type, the SOAP type, the word for specifying the input information, and the like. The nursing assistance information output unit 18 searches for and acquires the nursing information associated with the patient ID and including the character information of the search content. Accordingly, the nursing assistance information output unit 18 can acquire the nursing information matching the record content type, the SOAP type, the word for specifying the input information, and the like from the nursing information of the patient desired by the user. The nursing assistance information output unit 18 transmits a search result including at least the input information included in the acquired nursing information to the terminal 2. The search result may include all records (nurse ID, nursing type, SOAP type, and record content type) associated with the input information recorded as the nursing information. The above processing of the nursing assistance information output unit 18 is an example of an aspect of processing of outputting the nursing assistance information related to the input information of the patient to the output destination device based on the acquisition of the search request (designation information) for designating the identifier of the patient and the past input information regarding the patient from the output destination device (terminal).

The terminal 2 outputs the input information included in the search result to the search result display field 16B. The terminal 2 outputs information (nurse ID, nursing type, SOAP type, and record content type) other than the input information included in the search result to the detailed display field 16C. The user performs a pressing operation on the reading button B10. Upon detecting the pressing operation of the reading button B10, the terminal 2 performs processing of reading the text displayed in the search result display field 16B and outputs the read voice from a speaker. Accordingly, the user such as the nurse can listen to the past nursing information based on the search content while performing nursing work in hands-free. In a case where the user performs pressing operation on the stop button B11, the terminal 2 that detects the operation stops the reading processing.

While the example embodiment of the present invention has been described above, according to the above-mentioned processing, the handling alert is generated by utilizing the nursing information, and thus the information including the awareness of the nursing attendant for handling the patient can be efficiently notified of.

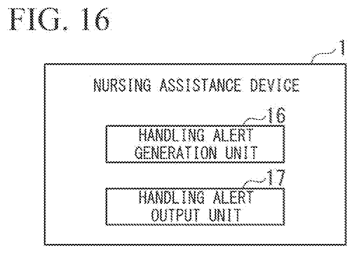

FIG. 16 is a diagram showing a nursing assistance device 1 according to another example embodiment.

As shown in FIG. 16, the nursing assistance device 1 includes at least a handling alert generation unit 16 and a handling alert output unit 17.

The handling alert generation unit 16 generates a handling alert regarding nursing based on the nursing information and a nursing handling rule of a patient.

The handling alert output unit 17 outputs a handling alert to an output destination device related to a nursing attendant of the patient.

The above-mentioned nursing assistance device 1 has a computer system inside. A procedure of the processing described above is stored in a computer-readable recording medium in the form of a program, and the processing is performed by the computer reading and executing this program.

The program may be for realizing some of the above-mentioned functions. The program may be a so-called difference file (difference program) that can realize the above-mentioned functions in combination with a program already recorded in the computer system.

This application is based upon and claims the benefit of priority from Japanese patent application No. 2018-199528, filed Oct. 23, 2018, the disclosure of which is incorporated herein in its entirety by reference.

### INDUSTRIAL APPLICABILITY

The present invention may be applied to a nursing assistance device, a nursing assistance method, and a recording medium.

### REFERENCE SYMBOLS

1: Nursing assistance device
2: Terminal
11: Control unit
12: Model generation unit
13: Acquisition unit
14: Type determination unit
15: Recording unit
16: Handling alert generation unit
17: Handling alert output unit
18: Nursing assistance information output unit
19: UI image generation unit
100: Nursing assistance system

1. A nursing assistance device comprising:
a handling alert generation unit that generates a handling alert related to nursing based on nursing information and a nursing handling rule of a patient; and
a handling alert output unit that outputs the handling alert to an output destination device related to a nursing attendant of the patient.

2. The nursing assistance device according to claim 1, wherein the handling alert generation unit specifies information related to a treatment included in the nursing information, and uses the nursing handling rule indicating a predetermined treatment corresponding to the treatment to generate a handling alert for prompting to perform the predetermined treatment.

3. The nursing assistance device according to claim 2,
wherein the nursing handling rule includes a rule indicating predetermined time associated with information indicating a specific medicine,
the nursing information includes first nursing information registered at a first point in time and second nursing information registered at a second point in time later than the first point in time, and
the handling alert generation unit generates the handling alert when the first and second nursing information includes information indicating the medicine and an interval between the first point in time and the second point in time is within the predetermined time.

4. The nursing assistance device according to any one of claims 1 to 3,
wherein the nursing information includes biological information of the patient,
the nursing handling rule includes a rule indicating a predetermined treatment corresponding to the biological information, and
the handling alert generation unit generates a handling alert for prompting to perform the predetermined treatment as the handling alert.

5. The nursing assistance device according to any one of claims 1 to 3, further comprising:
a nursing assistance information output unit that outputs nursing assistance information related to input information of the patient to the output destination device in response to acquisition of designation information for designating an identifier of the patient and past input information related to the patient from the output destination device.

6. The nursing assistance device according to any one of claims 1 to 4, further comprising:
an acquisition unit that acquires input information which serves as the nursing information from the output destination device that has started an operation based on communication with a predetermined communication device.

7. The nursing assistance device according to any one of claims 1 to 6, further comprising:
a severity degree calculation unit that calculates a severity degree calculated based on the nursing information; and
an image generation unit that generates an image having a different display aspect for each patient based on the severity degree, and outputs the image to the output destination device.

8. A nursing assistance method comprising:
generating a handling alert related to nursing based on nursing information and a nursing handling rule of a patient; and
outputting the handling alert to an output destination device related to a nursing attendant of the patient.

9. A recording medium storing a program for causing a computer to execute:
generating a handling alert related to nursing based on nursing information and a nursing handling rule of a patient; and
outputting the handling alert to an output destination device related to a nursing attendant of the patient.
